(19) United States
(12) Patent Application Publication
(10) Pub. No.: US 2026/0215874 A1
(43) Pub. Date: Jul. 30, 2026

(54) SYSTEM AND METHOD FOR LOCALIZATION OF A SURGICAL INSTRUMENT GUIDE

(71) Applicant: AiM Medical Robotics Inc., Worcester, MA (US)

(72) Inventors: Gregory S. Fischer, Needham, MA (US); Zhanyue Zhao, Shrewsbury, MA (US); Yang Wang, Shrewsbury, MA (US); Alexey D. Khotimsky, Westborough, MA (US)

(21) Appl. No.: 19/464,133

(22) Filed: Jan. 29, 2026

Related U.S. Application Data
(60) Provisional application No. 63/751,097, filed on Jan. 29, 2025.

(51) Int. Cl. A61B 90/11; A61B 90/00

(52) U.S. Cl. CPC A61B 90/11; A61B 90/39; A61B 2090/3937; A61B 2090/3954; A61B 2090/3966

(57) ABSTRACT

A guide is provided for holding an instrument having a longitudinal axis. The instrument guide comprises an integrated imaging fiducial for localization. The imaging fiducial including a plurality of substantially ring-shaped fiducial elements visible in at least one medical imaging modality. Each of the plurality of fiducial elements has a central axis that is coaxial with the instrument axis. At least one fiducial element is visible in at least one medical imaging modality and has a central axis that is not coaxial with the instrument axis.

### CROSS-REFERENCES

This application claims the benefit of U.S. Provisional Patent Application No. 63/751,097, filed Jan. 29, 2025, the contents of which is hereby incorporated by reference herein in its entirety for all purposes.

### BACKGROUND

A system and method for localization of a surgical instrument guide is described and, more particularly, localization of an instrument guide having integral fiducials for use of medical imaging in performing the localization,

Imaging of anatomical features can be useful in preparing for and performing surgical procedures. In some procedures it can be desirable to register the anatomy along with surgical instrumentation. This can ensure proper alignment and targeting of the instruments for the surgical procedure. The imaging process can use fiducial markers that can be recognized in medical imaging and are preoperatively placed in the anatomy of a patient. The fiducial markers can comprise elements fasteners having a geometry that is recognizable in imaging. Multiple fiducial markers are placed on the anatomy and can be used by a physician or surgeon for planning the surgical procedure, such as by providing a reference location for where an incision or cut can be located and/or a trajectory of an instrument.

There is a need for a guide for surgical instruments using fiducial markers to register the anatomy relative to the instrument and even the physical space of the operating room or any system associated with the referencing device.

For the foregoing reasons, there is a need for new systems and methods for localizing surgical instrumentation utilizing medical imaging. This includes localization of the position and orientation of robotically controlled modular instrument guides within imaging modalities, including 2D and 3D X-ray, Computed Tomography (CT), and Magnetic Resonance Imaging (MRI).

### SUMMARY

A guide is provided for holding an instrument having a longitudinal axis. The instrument guide comprises an integrated imaging fiducial for localization. The imaging fiducial includes a plurality of substantially ring-shaped fiducial elements visible in at least one medical imaging modality. Each of the plurality of fiducial elements has a central axis that is coaxial with the instrument axis. At least one fiducial element is visible in at least one medical imaging modality and has a central axis that is not coaxial with the instrument axis. The fiducial elements are visible in magnetic resonance imaging (MRI) or x-ray based imaging, including computed tomography (CT) and related techniques, or both. The fiducial elements are capable of being visualized and used to determine the instrument axis and anticipated trajectory relative to the medical imaging system.

In one aspect, the instrument guide further comprises means for determining the position and orientation of the fiducial and associated instrument holder with respect to the medical imaging system.

In another aspect, the instrument guide further comprises automatic segmentation of the fiducial elements individually, and fitting each of the fiducial elements to a known configuration relative to a coordinate system defined in the instrument holder of to a known model of the visible shape to determine the six degree-of-freedom (DOF) position and orientation of the instrument holder respect to the medical imaging system.

The instrument guide may further comprise a fiducial-integrated end effector designed to be part of a sterile single patient use kit.

The fiducial elements comprise hollow plastic, substantially ring-shaped components filled with a fluid visible in MRI, CT, or both MRI and CT. The fiducial elements are capable of being visualized as hyperintense in both MRI and CT. Substantially the same image processing localization software algorithm can be used to determine the position and orientation of the instrument holder in both MRI and CT.

In one feature, the fiducial elements are filled with fluid and visible in MRI, wherein an additional radiopaque material is used to make the fiducial elements visible in CT.

The instrument guide may further comprise a spacer ring between the MRI visible fiducial elements, wherein the spacer rings are visible in CT. At least part of the housing of the fluid-filled fiducial elements are visible in CT. The fiducial elements may comprise a material that is safe for use in MRI and does not introduce paramagnetic artifacts.

In yet another aspect, the inner diameter of the fiducial-integrated instrument guide is sufficient to pass a coaxial instrument holder configured for passing an instrument or a standard stereotactic neurosurgery instrumentation holder and frame adapter.

Another feature of the instrument guide is a linear adjustment parallel to the instrument axis. The linear adjustment may have discrete intervals or is continuously variable along a linear guide. The linear adjustment may be at least one of actuated and encoded.

In a further aspect, the fiducial-integrated instrument holder comprises an end effector of an actuated alignment device, and wherein the fiducial is used to determine position and orientation of the end effector relative to the medical imaging system. The position and orientation of the end effector relative to the medical imaging system in combination with kinematics information of the actuated alignment device are used to localize the coordinate system position and orientation of the actuated alignment device relative to that of the imaging system and the patient. The position and orientation of the fiducial-integrated instrument guide end effector is used for assessing the trajectory of the instrument after alignment and, if necessary, for repeated intraoperative registration of the alignment device to the imaging system. The position and orientation of the fiducial-integrated instrument guide end effector may also be used for identifying errors in alignment including alignment device positioning, registration errors, imaging distortion, target motion, or patient motion.

### BRIEF DESCRIPTION OF THE DRAWINGS

For a more complete understanding of the system and method for localization of a surgical instrument guide, reference should now be had to the embodiments shown in the accompanying drawings and described below.

In the drawings:

FIG. 1 is a schematic view of an embodiment of an instrument guide comprising a body and imaging-visible fiducial elements co-axial with an instrument axis.

FIG. 2 is a schematic view of another embodiment of an instrument guide comprising a body and imaging-visible fiducial elements co-axial with the instrument axis and additional imaging-visible fiducial elements in extensions to the body along orthogonal axes.

FIG. 3 is a schematic view showing an adapter coupled to the body of the instrument guide as shown in FIG. 1.

FIG. 4 is a schematic view showing an instrument-specific adapter coupled to the body of the instrument guide as shown in FIG. 2 and further comprising a cylindrical insert that is coaxial with the instrument and substantially matches the inside diameter of the instrument guide body and the outside diameter of the instrument.

FIG. 5 is a schematic view showing an instrument-specific adapter coupled to the body of the instrument guide configured to support a peel-away sheath cannula and allow insertion of an instrument such as a stylet through it to a specified depth.

FIG. 6 is a schematic view showing an embodiment of a keyed interface pattern cutout on the body of the instrument guide and a cylindrical thru hole that may mate with instrumentation including standard neurosurgical instrumentation and adapters.

FIG. 7 is a schematic view representative of a medical imaging scan volume showing imaging-visible fiducial elements as hyperintense hollow cylinders in their unique configuration that can be localized.

FIG. 8 is a schematic view representative of a projected instrument axis based on a localized position and orientation of the fiducial-integrated instrument guide from medical imaging overlaid on the patient's anatomical imaging.

FIG. 9 is a perspective view of one embodiment of a system for surgical instrument guide localization comprising an end effector of an actuated alignment device with a fiducial-integrated instrument guide, continuous linear adjustment along the instrument axis, and a cap with instrument-specific guide adapter.

FIG. 10 is a perspective view of another embodiment of a system for surgical instrument guide localization comprising an end effector of an actuated alignment device with a fiducial-integrated instrument guide, discrete linear adjustment along the instrument axis, and a keyed pattern for coupling with an instrument guide adapter.

FIG. 11 is a close-up perspective view with cover removed of one embodiment of the fiducial-integrated instrument guide.

FIG. 12 is a perspective view an alignment device coupled with an embodiment of a fiducial-integrated end effector and a set of compatible instrument guide adapters including a standard stepper device for stereotactic neurosurgery whose adapter is compatible with the instrument guide.

FIG. 13 is a close-up perspective view of the actuated alignment device as shown in FIG. 12 in a sterile drape and the fiducial-integrated instrument guide coupled through a sterile adapter.

FIG. 14 is a schematic view of a representative volumetric MRI scan of one embodiment of a fiducial for use in the localization guide.

FIG. 15 depicts a representative configuration of an embodiment of the localization guide wherein a robotic actuated alignment guide compatible with an MRI environment is coupled to a fiducial-integrated end effector instrument guide that is used for initial registration of the robot to the MRI imaging system and for subsequent confirmation of alignment and iterative re-targeting as needed.

FIG. 16 is a block diagram depicting a workflow for localizing the fiducial-integrated instrument guide and performing image-guided instrument alignment according an embodiment of the present invention.

### DESCRIPTION

Technologies are provided that can be used for localizing and targeting surgical instruments using medical imaging. The technologies include localization of the position and orientation of a surgical instrument guide comprising an imaging fiducial that is visible in one or more medical imaging modalities. In one embodiment, the imaging fiducial is integrated into an instrument guide and comprises a plurality of imaging fiducial elements or markers. The markers are composed of, or filled with, material visible in one or more medical imaging modalities. In one configuration, the imaging fiducial elements are substantially donut or toroidally-shaped or hollow cylindrically-shaped so that they show up as ring-like shapes in a 3D medical imaging modality. In an embodiment of the fiducial-integrated instrument guide, at least two such fiducial elements are positioned such that they are coaxial with a long primary axis of a surgical instrument such that an image of these fiducials is sufficient to virtually project the anticipated axis of the instrument through the guide.

In one configuration, the fiducial elements are visible by magnetic resonance imaging (MRI) and the fiducial is used to determine the up to 6 degree-of-freedom (6-DOF) position and orientation of a fiducial-integrated instrument guide relative to a coordinate system of an MRI scanner based on analyzing the representation of the fiducial elements in a set of MRI images. In another configuration, the fiducial elements are substantially radiopaque and visible using x-ray based imaging modalities, and in particular, visible in computed tomography (CT) and associated modalities, including cone beam computed tomography (CBCT) typically acquired on an intraoperative C-arm fluoroscopy system, intraoperative CT (ioCT), and O-arm-like portable CT systems. Similarly, the fiducial and associated instrument guide are localized in up to 6-DOF relative to a coordinate system of the CT scanner or other tomographic imaging system. In one embodiment, the fiducial elements are hollow and filled with a fluid that is visible in both MRI and CT providing for a multi-modality fiducial wherein the images in both modalities show hyperintensity at the fiducial element locations and show substantially the same fiducial pattern, thus enabling substantially the same image processing to be used to determine the position and orientation of the fiducial and associated instrument guide relative to the imaging modality. The fluid used may be iodine-based, such as Lugol's solution which has been shown to be visible in both MRI and CT. In another embodiment, fiducial elements may alternate with visibility in different imaging modalities, for example alternating a hollow ring filled with an MRI contrast agent with a solid substantially radiopaque ring.

In one embodiment, the fiducial-integrated instrument guide comprises a hollow substantially tubular shape along the primary axis of the instrument that passes through a central opening of two or more co-axial fiducial elements. Modular instrument adapters and guide sleeves are configured to pass through this central opening and lock into the instrument guide that hold instruments aligned with, and coaxial with, the cylindrical opening, adapters, guide sleeves, and fiducial elements along that axis. The use of fiducial elements coaxial with the surgical instrument enables the instrument to be localized in the medical imaging modality directly while avoiding sources of error such as unknown offsets. Further, this approach enables virtual projection or prediction of the path an instrument would follow should it be inserted along that path—this can be visualized and overlaid in medical imaging prior to delivery of the instrument into the patient and/or iteratively as it is inserted to a target location. In one use case, the fiducial of the fiducial-integrated instrument guide is imaged in MRI in the same volume as a patient's anatomy, and the trajectory or path of the instrument guide's axis is able to be displayed on the patient imaging to ensure it is aligned to target. This enables confirmation before delivering an instrument, or can be used for iterative correction of alignment until the instrument axis is sufficiently aligned with the intended trajectory. This is of particular interest in neurosurgery interventions, wherein the trajectory of the instrument guide can be overlaid to determine if it will pass through the intended burr hole cleanly, avoid critical structures, and reach the desired target, despite the potential for brain shift, swelling, registration error, patient motion, or other typical causes of error. This can be used in MRI, CT-based modalities, and other 3D imaging modalities, including ultrasound and photoacoustic imaging. In another use case, one or more projection images, such as from x-ray fluoroscopy, may be acquired and the 3D shape of the fiducial reconstructed to determine its position and orientation.

In one embodiment, the fiducial-integrated instrument guide is coupled to the end effector of an alignment device such as, but not limited to, a stereotactic frame or passive alignment arm. In a further embodiment, the alignment device is an actuated device such as a surgical robot. In one configuration, the fiducial-integrated instrument guide is coupled to the end effector of an actuated alignment device resembling an arc-like stereotactic frame, and that robotic actuated frame is configured to be compatible with operating in MRI and CT environments. The determination of the 6-DOF position and orientation of the fiducial integrated into the instrument guide is used to determine where the alignment guide is relative to the medical imaging system, and thus also a patient and an associated surgical plan. In one embodiment, the actuated alignment guide is positioned in an arbitrary but known configuration, its fiducial imaged with the medical imaging modality, the 6-DOF position and orientation of the fiducial determined by software, and a transformation based on the known alignment guide's configuration applied to determine the position and orientation of the actuated alignment guide and its internal coordinate system to that of the medical imaging system. This enables localization of the alignment guide device and the instrument with a single localization fiducial. After the alignment guide is moved into a desired targeting position, the fiducial is imaged again and its position and orientation identified by the software. This step enables confirmation of alignment relative to the surgical plan and also virtual projection of the instrument axis through the patient's imaged anatomy to confirm an ideal trajectory, which may have changed since the initial planning due to tissue motion, alignment errors, registration errors or a host of other possibilities. As necessary, the alignment guide may be iteratively adjusted and the fiducial imaged until sufficient targeting is confirmed and the instrument is delivered along the axis of the instrument guide through a co-axial adapter or guide sleeve. In one configuration, every time the fiducial of the fiducial-integrated instrument guide end effector of the alignment guide is localized, that information along with the current configuration (e.g. forward kinematics) of the alignment guide is used to perform a re-registration to determine and either confirm and/or update the position and orientation of the actuate alignment guide's coordinate system relative to the medical imaging system's coordinate system.

One use case for the system and method for localization of a surgical instrument guide is for supporting robot-assisted, MRI-guided stereotactic neurosurgery, including deep brain stimulation (DBS) neurostimulator lead placement, stereo electroencephalography (SEEG) electrode placement, biopsy, cannula placement, delivery of drains and shunts, guiding and holding instruments such as endoscopes and drills, ablation or other localized heating and/or vibration including laser interstitial thermal therapy (LITT) and needle-based therapeutic ultrasound (NBTU), and targeted drug delivery including intratumoral chemotherapy agents, gene therapy, and cell therapy. These are just a sample of potential use cases wherein the instrument guide as described herein supports localization and guidance of the instruments.

The surgical instrument guide also supports the use in one or more imaging modalities including MRI and/or CT-based imaging, and can be used in a dedicated medical imaging suite, intraoperative imaging suite, traditional operating room, or other location including remote locations and terrestrial and aerospace vehicles. The system and method for localization of a surgical instrument guide is not limited to cranial anatomy and may be used in a variety of locations in the body including, but not limited to: head and neck, spine, thoracic, abdominal, gynecological, urological, and other procedures. The present invention is not only limited to medical applications, and its use may be enjoyed in other use cases such as scientific and industrial applications where localization of an instrument or tool is desirable relative to an imaging system.

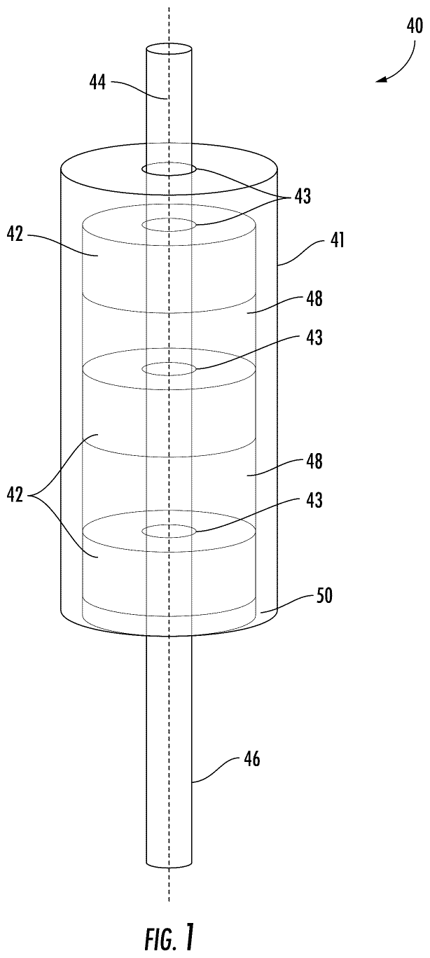

Referring now to the drawings, wherein like reference numerals indicate the same of similar elements throughout the several views, a fiducial-integrated instrument guide is shown in FIG. 1 and generally designated at 40. The instrument guide 40 comprises a body 41 and imaging-visible fiducial elements 42 co-axial with the instrument axis 44. An instrument 46 passes through central guide holes or sleeves 43. In one embodiment, the imaging fiducials 42 are integrated into an instrument guide 40 and comprise a plurality of imaging fiducial elements or markers 42. The markers are composed of, or filled with, material visible in one or more medical imaging modalities. In one configuration, the fiducial elements 42 are visible in magnetic resonance imaging (MRI) and the fiducial is used to determine the up to 6 degree-of-freedom (6-DOF) position and orientation of a fiducial-integrated instrument guide 40 relative to a coordinate system of an MRI scanner based on analyzing the representation of the fiducial elements 42 in a set of MRI images. In some cases, 5-DOF is sufficient because the instrument is rotationally symmetric and therefore rotation angle about its axis is unnecessary. Such 5-DOF localization can be performed with a set of coaxial fiducial elements 42 alone as shown in FIG. 1.

In an embodiment, the fiducial elements 42 are substantially radiopaque and visible using x-ray based imaging modalities, and in particular visible in computed tomography (CT) and associated modalities including cone beam computed tomography (CBCT) typically acquired on an intraoperative C-arm fluoroscopy system, intraoperative CT (ioCT), and O-arm-like portable 3D X-ray or CT systems. Similarly, the fiducial 42 and associated instrument guide 40 are localized in up to 6-DOF relative to a coordinate system of the CT scanner or other tomographic imaging system. For the purposes of this application, CT encompasses all tomographic 3D X-ray based imaging modalities.

In a configuration, the fiducial elements 42 are hollow and filled with a fluid that is visible in both MRI and CT providing for a multi-modality fiducial wherein the images in both modalities show changes in intensity relative to the background (such as both showing bright hyperintensity at the fiducial element locations in both modalities) and show substantially the same fiducial pattern, thus enabling substantially the same image processing to be used to determine the position and orientation of the fiducial 42 and associated instrument guide 40 relative to the imaging modality. The fluid used may be iodine-based, such as Lugol's solution, which has been shown to be visible in both MRI and CT. In another configuration, fiducial elements 42 may alternate with visibility in different imaging modalities, for example alternating a hollow ring filled with an MRI contrast agent with a solid substantially radiopaque ring. In one embodiment, the fluid is visible in one modality (e.g. MRI) and the body of the ring is visible in another modality (e.g. CT). The fiducial elements 42 may may be separated by spacers 48, which may all be the same spacing, or spacers 48 may be intentionally different heights to introduce asymmetry enhancing reliable image processing and registration. A cap 50 may hold the elements 42 and spacers 48 inside body 41.

In a configuration as shown in FIG. 1, the imaging fiducial elements 42 are substantially donut or toroidally-shaped or hollow cylindrically-shaped so that they show up as ring-like shapes in a 3D medical imaging modality. In an embodiment of the fiducial-integrated instrument guide 40, at least two such fiducial elements 42 are positioned such that they are coaxial with the long primary axis 44 of the surgical instrument 46 such that an image of these fiducials 42 is sufficient to virtually project the anticipated axis of the instrument 46 through the guide 40.

In an alternate configuration, the body 41 of the instrument guide 40 has a cavity which is filled with a fluid visible in the desired one or more imaging modalities, and a stepped insert is placed into the cavity to displace the liquid in such a way that it generates substantially the same pattern in the medical imaging as stacked fluid-filled rings 42 and spacers 48.

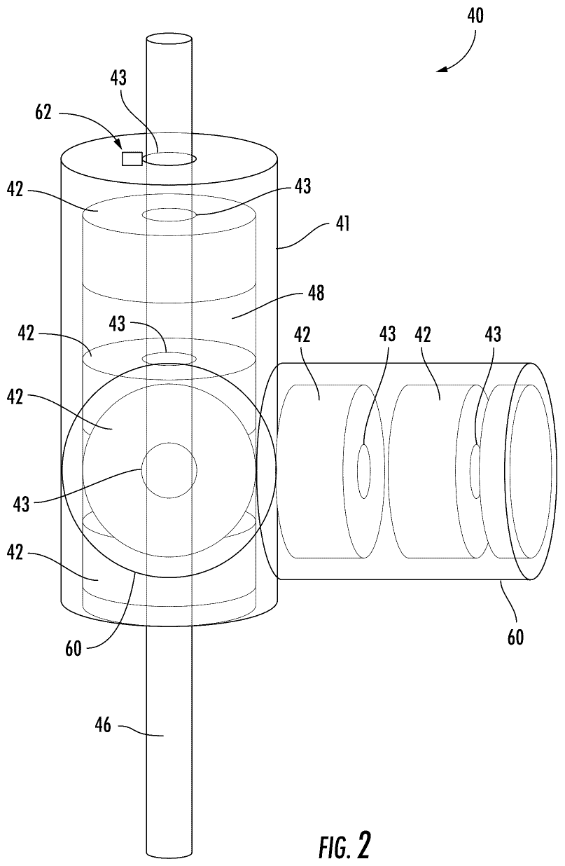

Referring to FIG. 2, a fiducial-integrated instrument guide 40 comprises a body 41 and imaging-visible fiducial elements 42 co-axial with the instrument axis 44 and additional imaging-visible fiducial elements 42 in an extension 60 to the body along an additional orthogonal axis. Two or more, and preferably at least three fiducial elements 42 are disposed along the instrument axis 44 alone and enable up to 5-DOF localization. To extend to 6-DOF, one or more additional fiducial elements 42 are included in the body 41 of the instrument guide 40. Many configurations of such fiducial elements 42 may be used, typically ensuring at least three total fiducial elements are visible, and it is understood that the user is not restricted to the number or configuration of said fiducial elements 42. In one embodiment, at least one, and preferably two fiducial elements 42 are placed on an axis orthogonal to the instrument axis 44. To make the localization more robust, an additional fiducial element 42 may be placed out of the plane of the previously noted fiducial elements 42, and the user is not restricted to the number or configuration of said additional fiducial element(s). In one embodiment, at least one fiducial element 42 is placed on an axis mutually orthogonal to the previously noted two axes. Since the fiducial 42 can be localized in 6-DOF including instrument rotation, a key 62 or similar means for locating and locking instrument rotation about the instrument axis 44 is incorporated. While typically instrumentation 46 is passed along the primary axis 44 of the instrument which is aligned with the instrument guide 40, where the term coaxial is used with respect to instruments, this invention also includes the use of offset adapters such as a ben-gun array or multi-lumen device that enables multiple trajectories parallel to, but slightly offset from, the primary axis 44.

A unique number of fiducial elements 42, such as four fiducial elements coaxial with the instrument axis 44, two fiducial elements 42 coaxial with an axis 60 orthogonal to the instrument axis, and one fiducial element 42 coaxial with a mutually orthogonal and intersecting axis provide for a robust localization and minimization of errors and confusion by clearly identifying the principal axes. This configuration is also readily identified and localized manually in imaging system software and traditional surgical planning and navigation software without the need for specialized image processing algorithms. The present description includes the design of the pattern of the fiducial and the algorithm for localizing it in medical imaging.

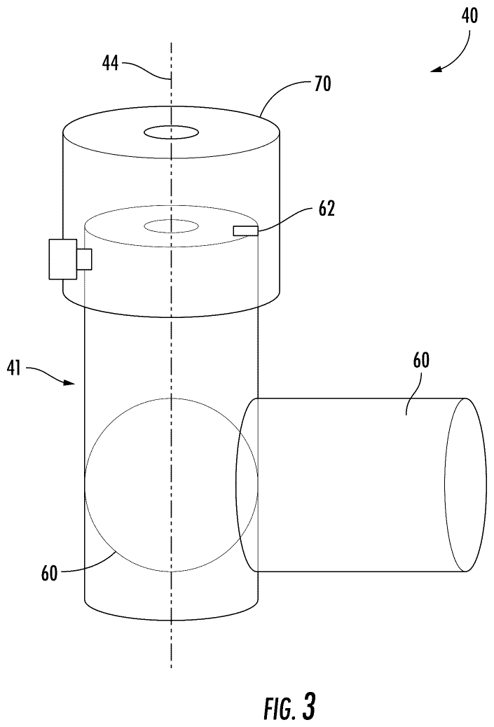

FIG. 3 shows an adapter 70 coupled to the body 41 of the fiducial-integrated instrument guide 40. A variety of caps or adapters 70 may be used to support the use of a variety of instrumentation. Some examples of instrumentation for stereotactic neurosurgery that can be held and guided by the cap or other adapter 70 of the fiducial-integrated instrument guide include, but are not limited to: deep brain stimulation (DBS) neurostimulator leads, stereo electroencephalography (SEEG) electrode placement, microelectrode recording electrodes and arrays (MER), microdrive steppers, biopsy devices including needles and aspirators, cannulas including split peel-away cannulas and rigid cannulas, drains and shunts, manual and powered drills and drill guides, screws or bolts and associated drivers, tracked navigation pointers and rigid bodies, endoscopes, retractors, ablation or other localized heating and/or vibration inducing devices including laser interstitial thermal therapy (LITT) and needle-based therapeutic ultrasound (NBTU), and targeted drug delivery devices including those for intra-tumoral chemotherapy agents, gene therapy, and cell therapy. The cap or adapter 70 is keyed 62 to the body 41 of the instrument guide 40 and may be repeatably attached in a known orientation about the axis and with a repeatable height along the axis.

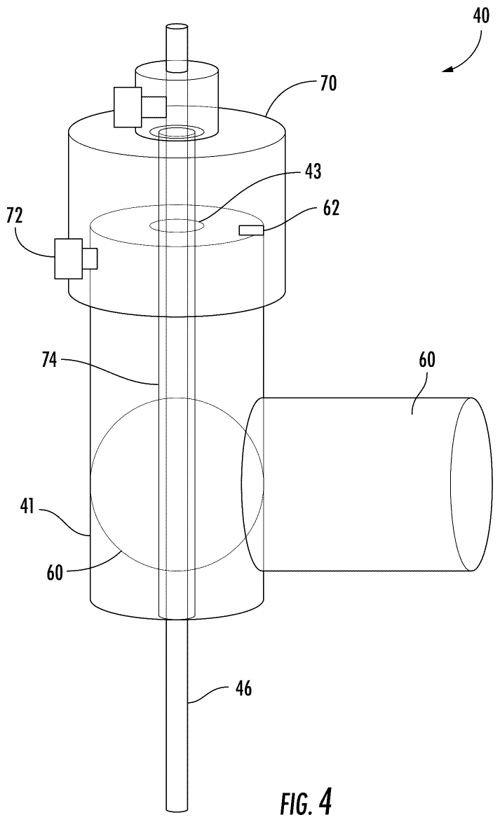

FIG. 4 shows an instrument-specific adapter 70 coupled to the body 41 of the instrument guide 40 comprising a cylindrical insert that is coaxial with the instrument 46 and, substantially matches the inside diameter of the instrument guide body 41 to the outside diameter of the instrument 46. The inside diameter of the instrument adapters 70 may be configured to specifically match the outer diameter of the instrumentation, and a set of such adapters may be made available for common instrument types and/or diameters. Guide sleeves 74 may extend through a central tubular cavity 43 in the body 41 of the instrument guide 40 and guide the instrument 46 along an extended portion of its length, and may extend beyond the distal end of the instrument guide body 41. These guide sleeves 74 may be tubes of low friction plastic or ceramic material, such as for passing needle and cannula-like instruments, or may be made of a more rugged material such as composite or metal when used with cutting instruments such as drills. Adapters 70 are configured with a known height offset such that one can readily identify the necessary insertion depth to the target relative to the proximal portion (i.e. top as shown) of the instrument guide 40. This can be used to determine the location of a depth stop along the axis of the instrument 46 on the proximal side of the instrument guide 40, or to determine how much actuation should be provided for an actuated or otherwise adjusted instrument insertion.

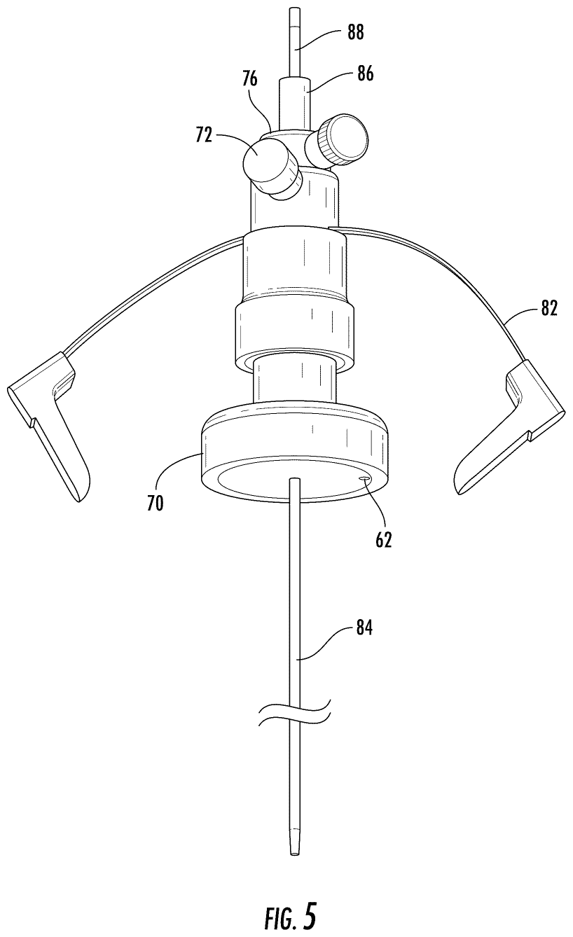

FIG. 5 depicts an example instrument-specific adapter 70 that can be coupled to the body 41 of the instrument guide 40. A cap 50 or other adapter 70, optionally with a key 62, couples to the instrument guide body 41. This example is configured to support a peel-away sheath cannula 82 and allows insertion of an instrument such as a stylet 46 through it to a specified depth. This is a typical configuration used for delivery of deep brain stimulation (DBS) neurostimulation electrodes in specific anatomical targets in the brain, where the stylet 88 is inserted into the brain with the peel-away cannula, the stylet is replaced with the electrode, and the cannula ends 82 are peeled back pulling out the remaining cannula 84 leaving the electrode in place. Depth stops 72 and 76 may be used to set the cannula and inserted instrumentation depths. In one configuration, rigid cannulas are used instead of peel-away cannulas, and they are pulled in and out along the instrument axis 44 to deliver and then leave behind, respectively, the electrode or other instrumentation. Other adapters may be configured for other instrument types and/or diameters. In one configuration, a single-patient sterile kit comprises the fiducial-integrated instrument guide and a set of typical adapters.

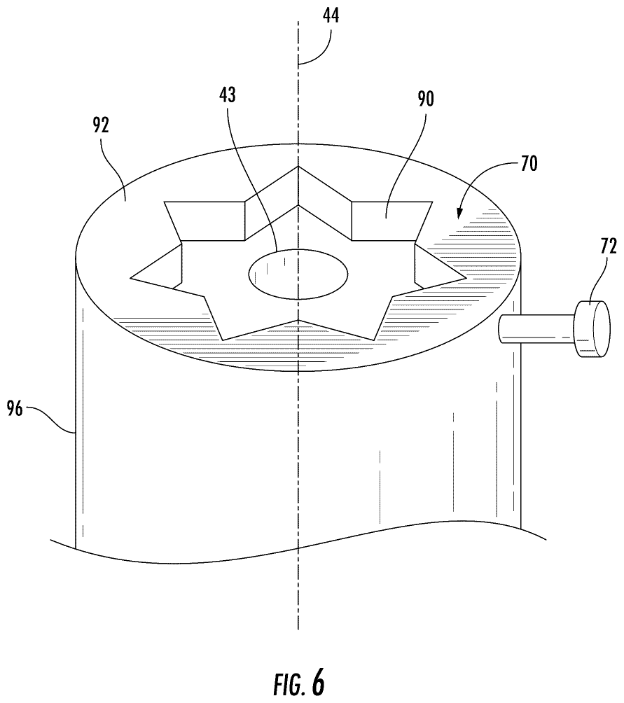

FIG. 6 shows a keyed interface pattern 90 that may be formed into an adapter cap 96 or directly on the body of the instrument guide 40. In one embodiment, the interface pattern is a recess 90 into a top surface 92 and further comprises a cylindrical thru hole 43 and a means of fixation such as a set screw 72. The interface pattern 90 is shown as a star-like cutout, but the invention is not limited to this shape. Other embodiments include various standard and non-standard interface patterns. The interface pattern may comprise a recess in the material, a protrusion, or both, and it may be made by various means including molding, machining, and additive manufacturing. In one configuration, a cap or adapter 70 mates with the interface pattern 90 and has a surface that mates flush with the top surface 92. The cap or adapter 70 comprises an inverted pattern similar to that of the interface pattern 90 that mates within the interface pattern 90. The mating provides for rotation and translational mating consistency between the instrument, or its associated adapter or cap, and the fiducial-integrated instrument guide 40. The cylindrical surface 43 ensures that the primary axis of a substantially cylindrical instrument or adapter is aligned with the axis 44. The present invention does not preclude the use of curved or flexible instrumentation within the instrument guide 40 that do not have a fixed axis along their entire length, but will be aligned at the point in which they are guided within the instrument guide 40.

One configuration is intended to enable the fiducial-integrated instrument guide 40 to mate with instrumentation and adapters. This includes, but is not limited to, standard neurosurgical instrumentation and adapters, such as the variety of commercially available stereotactic frame adapters and associated instrumentation. In one embodiment, the body of the fiducial-integrated instrument holder is configured to couple with a standard commercially available microdrive such as the manual and motorized FHC STarDrive. The fiducial-integrated instrument holder 40 may be configured to be compatible with all standard instrumentation intended for use with traditional stereotactic frames such as the Leksell G-Frame and Vantage. Similarly, the fiducial integrated instrument guide 40 may be configured to support a plethora of existing instrumentation from robotic and manual alignment devices, including instruments, adapters, guides, and implants.

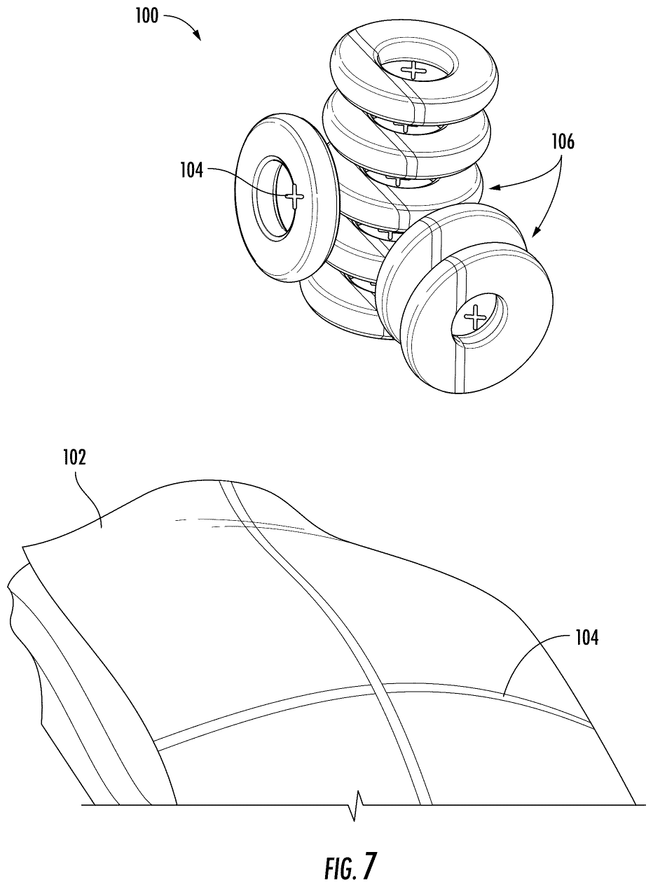

FIG. 7 is a representative medical imaging scan volume 100 in a surgical navigation software, in this case shown in MRI. It shows the imaging-visible fiducial elements 42 as bright white hyperintense ring-like shapes 106 in their unique configuration, which would typically be shown on a dark background, and in some cases alongside imaging of patient anatomy 102. Imaging of the fiducial-integrated instrument guide 40 may have a small tight field of view directed to the region of the fiducial elements 42, or may be broader, sometimes including visualization of the patient anatomy 102. This visualization would appear similarly in CT.

Shown here is the visualization of one embodiment of the imaging-visible fiducial elements 42 filled with an imaging visible fluid. The fluid may be visible in MRI, CT, or both. It may show as hyperintense, hypointense, or otherwise providing contrast to nearby regions. Each fiducial element can be localized individually, identified, and its center or origin 104 determined with respect to the coordinate system of the medical imaging system as shown.

The known configuration of the fiducial elements 42 relative to a coordinate frame and origin on the fiducial-integrated instrument guide 40 and the measured location of the origins in the medical imaging are used to calculate the position and orientation of the instrument guide 40 relative to imaging system. This may be performed with a least squares point cloud to point cloud registration, or other technique. In another approach, rather than individually identifying the centroid of each fiducial element, a 3D model based fit of all or a subset of the fiducial clements 42 may be used to match the known 3D model of the fiducial elements 42 to their corresponding imaging representations 106. A 3D model-based approach for either the individual elements or the full fiducial may provide greater robustness to air bubbles or otherwise missing features such as a cropped field of view because it does not rely exclusively on the center of mass of the associated pixels 106 for a given fiducial element 42 in imaging volume 100.

Localization of donut-shaped individual fiducial elements 42 is beneficial because many surgical navigation software platforms can readily find and determine the origin of such substantially donut-shaped markers. A variety of means for identifying the markers and finding their origins may be used, including template-based approaches to find donut-like hyperintense objects and then find the centroid of them. Template based techniques may be used to find the origin of the hyperintense donut-like objects in a way that is more robust to air bubbles, noise, and other imaging artifacts. In another configuration, a template based model may be used to directly identify the complete shape of the fiducial rather than individually identifying the location of individual fiducial elements or markers. The present invention is not limited only to these image processing and registration approaches, and may utilize other such techniques known to one skilled in the art.

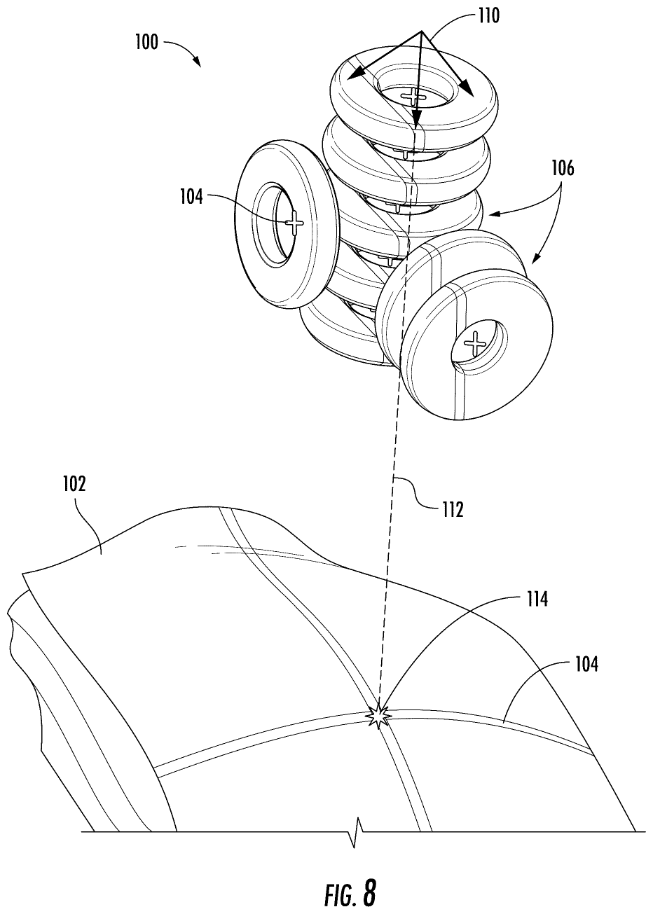

FIG. 8 is a representation of a projected instrument axis 112 based on the localized position and orientation represented as coordinate frame 110 of the fiducial-integrated instrument guide 40 from medical imaging 100. It is shown overlaid on the patient's anatomical imaging with anatomy shown as 102. Patient anatomy 102 may be acquired in the same imaging volume as the fiducial elements 106, or may be acquired in another imaging volume with a different field of view or other varied parameters. The pose 110 and axis 112 may be overlaid on anatomical imaging to perform initial registration, create a surgical plan, visualize a surgical plan, confirm instrument alignment prior to delivery, confirm instrument alignment or placement after delivery, or iteratively replan and optimize alignment and delivery.

Surgical navigation software or standard software on the medical imaging system may be used to determine the instrument axis 112 and assess its alignment with imaging of the target anatomy 102. Target anatomy 102 of the patient may be imaged in the same set of medical images as the fiducial 42, or may be acquired in a separate set and merged. The former allows a single image volume to be acquired. The latter allows tight field of view with optimized parameters for fiducial localization and optimal field of view and scan parameters for visualizing target anatomy, which can still be overlaid and shown at the same time. The instrument axis can be readily identified manually in standard medical imaging visualization software due to the concentric coaxial fiducial rings along the instrument axis and orthogonal axes comprising additional fiducial elements. Software may be used to automatically determine the position and orientation of the instrument axis and virtually project that on images of the patient anatomy and/or surgical plan.

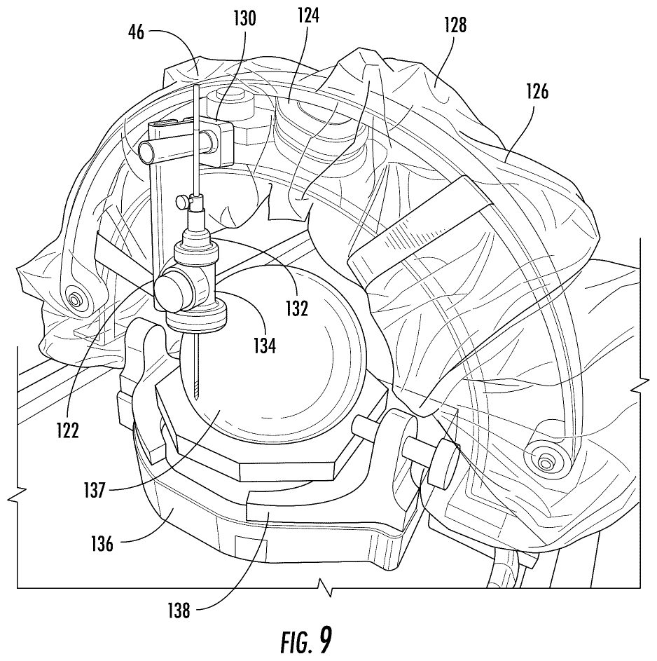

FIG. 9 depicts one embodiment of the system and method for localization of a surgical instrument guide comprising an end effector 130 of an actuated alignment device 126 with a fiducial-integrated instrument guide 134. A means for continuous linear adjustment 122 along the axis of the instrument 46 allows the adjustment of the height of the base of the fiducial integrated instrument guide 134 above the surface of the patient anatomy. In this embodiment, the patient anatomy is the head of a patient 137 and the actuated alignment device 126 is a robotic stereotactic frame capable of operating in both MRI and CT environments while maintaining safety and image quality. Patient head 137 is fixed in a head fixation device 138, which is integrated into a base platform 136. The base platform 136 couples to the bed of the imaging system (e.g. MRI bed or CT bed) or an operating room table. The actuated alignment device 126 is removably attachable to the base platform 136. The actuated alignment guide is shown wrapped in a tubular sterile drape 128. End effector 130 and its associated components may be part of a pre-sterilized reusable kit or part of a single use presterilized kit, or a combination thereof. An adapter or cap 132 couples to the fiducial-integrated instrument guide 134 and may be design to hold and guide to a specific instrument 46.

In one configuration, the fiducial-integrated instrument guide 134 acts as an end effector 130 that is coupled to the carriage of a robotic stereotactic frame 126. It is coupled through a sterile drape 128 with a sterile adapter that is either attached between the instrument guide and the drape to enable adjustment of offset height, or the sterile adapter is an integral part of the sterile drape.

The actuated alignment device has a known configuration such that the forward kinematics provide the known position and orientation of the fiducial-integrated instrument guide 134 with respect to the base coordinate system of the alignment device. When imaged with the imaging modality (such as, but not limited to, MRI and/or CT), software provides the 6-DOF position and orientation of the fiducial-integrated instrument guide with respect to the medical imaging system. Combining these two transformations enables one to determine the 6-DOF position and orientation of the actuated alignment device relative to the medical imaging system. This step is referred to registration and can be performed in arbitrary pose of the actuated alignment device 126, and can be repeated as often as desired. Registration is a necessary step to enable desired instrument trajectories planned based on medical images of the patient to be converted into desired poses of the actuated alignment guide. The alignment guide moves to the desired pose and images of the fiducial may be acquired to confirm alignment as intended since necessary alignment can be iterated until satisfactory. Each fiducial imaging cycle can be used to update the registration or check that registration has not changed. Coupling repeated imaging of the fiducial and the patient anatomy allows for tracking of the instrumentation, the target inside the patient, and potential obstacles so that the surgical plan can be updated as necessary and ensure a safe and accurate procedure is performed.

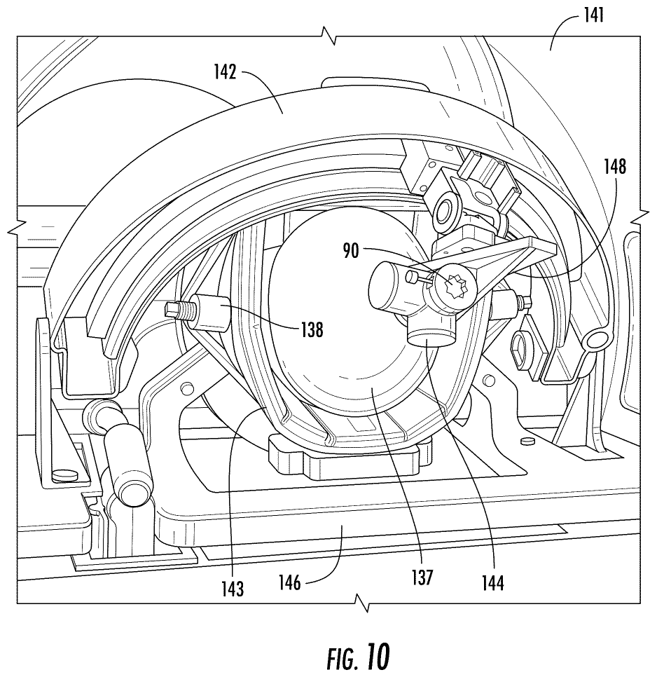

FIG. 10 depicts one embodiment of the system and method for localization of a surgical instrument guide comprising a fiducial integrated instrument guide 40 as an end effector of an actuated alignment device 142. The fiducial-integrated instrument guide 40 has discrete linear adjustment of the offset 148 along the instrument axis to adjust the height of the instrument guide, and a keyed pattern 90 for coupling with an instrument, instrument guide, cap, adapter, or other available instrumentation. Height adjustment 148 allows optimizing the distance between the distal end of the instrument guide and the patient anatomy. In some cases, this should be minimized for maximal stability and in other cases expanded for increased access and visibility at the operator's discretion. This embodiment also depicts the actuated alignment device (i.e. robotic stereotactic frame) 142 coupled to a base platform 146 that is fixed to the imaging system's bed. The example base platform 142 here is shown mated with a Siemens MRI system by matching the base platform with the T-slots on the top surface of the bed. Various adapters and base platforms may be used for other imaging system models, vendors, and modalities. The base platform further comprises a means for fixation 138 of the patient's head 137 relative to the alignment device 142.

The entire system shown in FIG. 10 is configured to fit and operate within the tubular bore 141 of an MRI scanner. Also shown is an MRI coil that fits within the fixation device 138 between said fixation device and the head 137. Integrating an MR imaging coil can improve image quality in MRI, and doing so much ensure maintained access to the patient for surgery and clearance of the moving alignment device 142.

The registration step determines the position and orientation of the coordinate system on the alignment device's base relative to that of the medical imaging system, the medical images, and the patient. For the case of neurosurgery, when coupling with MRI images, brain shift can be accounted for to adapt the surgical plan to reach a specific intracranial soft tissue target, and vasculature can be identified to avoid inadvertent puncture.

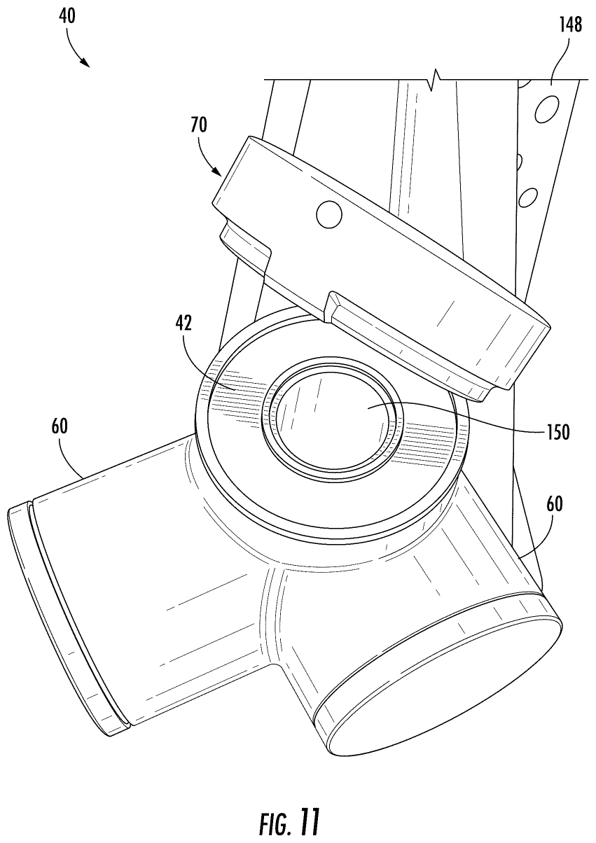

FIG. 11 provides a close-up view with cover 70 removed of one embodiment of the fiducial-integrated instrument guide 40. Shown is an example of a donut-shaped fiducial element 42. This example fiducial element 42 is filled with an MRI contrast fluid and has a substantially square cross-section, however round or other cross-sectional shapes may also be used. This embodiment has four fiducial elements 42 coaxial with the instrument axis (aligned with the tubular interface 150 for instrument adapter shown), a stack of two fiducial elements coaxial with an axis orthogonal to the instrument axis in one of the extensions 60, and one fiducial element coaxial with a mutually orthogonal and intersecting axis in another extension 60. In this embodiment, the axes for the two extension arms 60 are mutually orthogonal (i.e. the axis of all three stacks of fiducial elements 42 are substantially orthogonal) and offset along the primary instrument axis to improve robustness of image processing algorithms. The spacing between fiducial elements 42, especially along the primary instrument axis 44 which is coaxial with cylindrical surface 150, may be varied to avoid symmetry and clearly demarcate the distal and proximal ends in imaging. While the presented example uses a set of all substantially identical fiducial elements 42, which provides for the advantage of reduced assembly and manufacturing complexity, such a configuration is not required and the fiducial elements 42 may take on two or more unique shapes within the same fiducial integrated instrument guide 40.

The configuration in FIG. 11 provides for robust localization and minimization of errors and confusion by clearly identifying the principal axes. This configuration is also readily identified and localized manually in imaging system software and traditional surgical planning and navigation software without the need for specialized image processing algorithms. However, the present invention does not require all three axes to be mutually orthogonal, does not require an offset between extensions, and is not restricted to any specific number or configuration of financial elements 42 or spaces between.

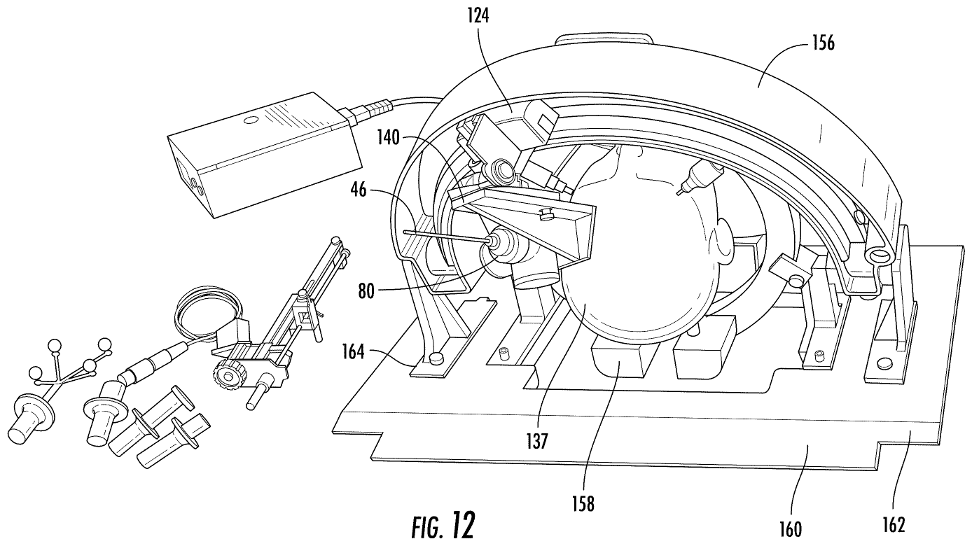

FIG. 12 depicts an embodiment of an actuated alignment device 156 (arc-shaped robotic stereotactic frame) coupled with an example fiducial-integrated instrument guide 40 acting as an end effector of the alignment device 156, coupled to a moving component (carriage) of the actuated alignment device 156. It also depicts a set of compatible instrument guide adapters 80 for holding a variety of instruments 46. Instrument guide adapters 80 may include adapters for standard stepper devices for stereotactic neurosurgery whose adapter is compatible with the instrument guide and adapters for other various instrument types and diameters including for tracked navigation, drills, peel-away sheath split cannulas, and pointers. In the example, the adapters have a standard star-like key pattern which mates with the pattern on the body of the instrument guide to allow a variety of adapters and instrumentation to be attached to the fiducial integrated instrument guide 40. The offset height of the instrument guide may be adjusted to set the height of the instrument guide as desired above the surface of the head 137, and that offset information is included in the kinematics calculations when performing registration. The head 137 is fixed to base platform 160 through head fixation system 158. The base platform 160 couples to the bed of an imaging system or operating room table using appropriate features 162. The actuated alignment guide 156 is removable coupled to the base platform using coupler 164. The coupler can be readily attached and detached during, before, and after a procedure.

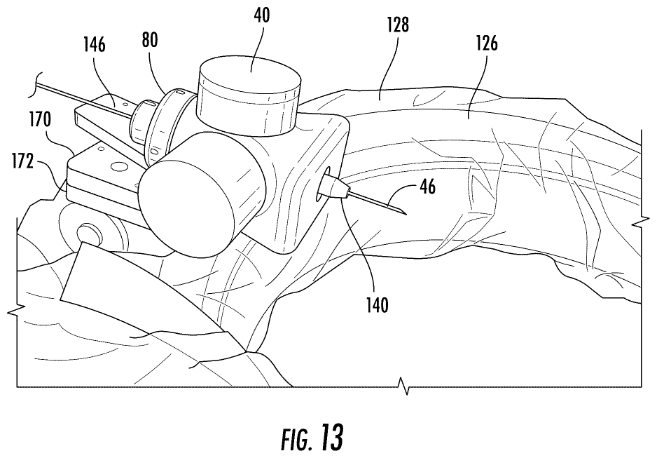

FIG. 13 is depicts an actuated alignment device 126 in a sterile drape 128. A fiducial-integrated instrument guide 40 is coupled through a sterile adapter 170. The arc-like actuated alignment device is covered in a substantially tubular sterile drape 128. The fiducial-integrated instrument guide 40 is a sterile component and attached as an end effector to the alignment device 126. The fiducial integrated instrument guide has an adapter 80 that couples through a star-like keyed interface and guides an instrument guide sleeve 140. The guide 140, which may be integrated into the adapter 80 or a separate part supported by adapter 80, serves as a guide for instrument 46.

To enable adjustment of the offset height using the adjustable mount 148 while maintaining sterility, a sterile adapter 170 is used between the drape and the instrument guide. In one example, the adjustable mounting interface 18 between the fiducial-integrated instrument guide 40 and the sterile adapter 170 comprises 2 pins and one thumb screw, however, alternate variations of this interface may be implemented by using techniques known to one skilled in the art. In one example, the sterile adapter 170 is coupled to the distal interface portion of the robot 172 using two pins and a screw, however, alternate variations of this interface may be implemented by using techniques known to one skilled in the art. In one embodiment, this sterile adapter 170 is an integral part of the drape rather than a stand-alone sterile part of the sterile kit, and in another embodiment it is a stand-alone part in the sterile kit. In one embodiment, the fiducial-integrated instrument guide 40 and its accessories are configured as single-patient disposable sterile components and prepared in a per-patient sterile kit. In an alternate embodiment, some or all of these parts are configured as a durable or limited lifetime sterilizable reusable equipment.

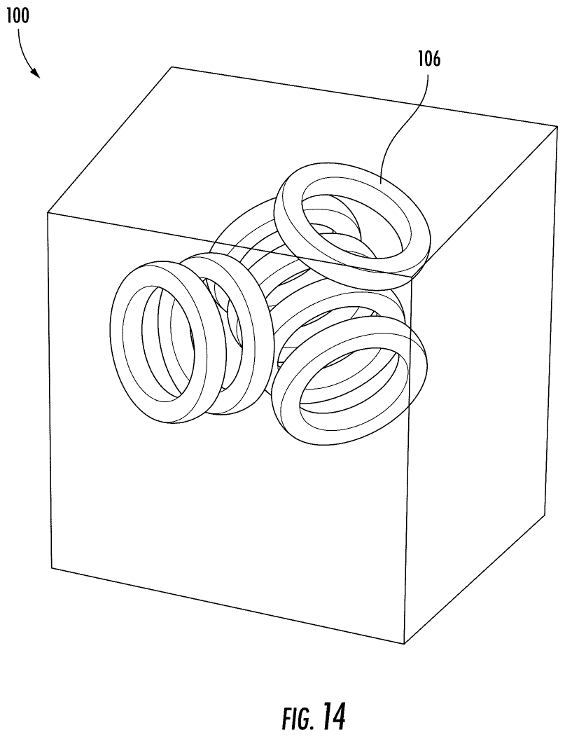

FIG. 14 shows a representative MRI scan imaging volume 100 accommodating one configuration of the fiducial 42. It shows the hyperintense pattern 106 visible in MRI of the fiducial shown in FIGS. 10-13. This depicts how it is visualized in 3D in standard GE MRI console software. The fiducial 42 would appear similarly in other medical imaging system interfaces, surgical panning and navigation interfaces, and custom software for control of this platform. Accordingly, the present invention comprises a multi-modality imaging fiducial that would show up similarly in both MRI and CT-based imaging techniques, and can use substantially the same image processing and registration calculation techniques with the same fiducial in the different imaging modalities.

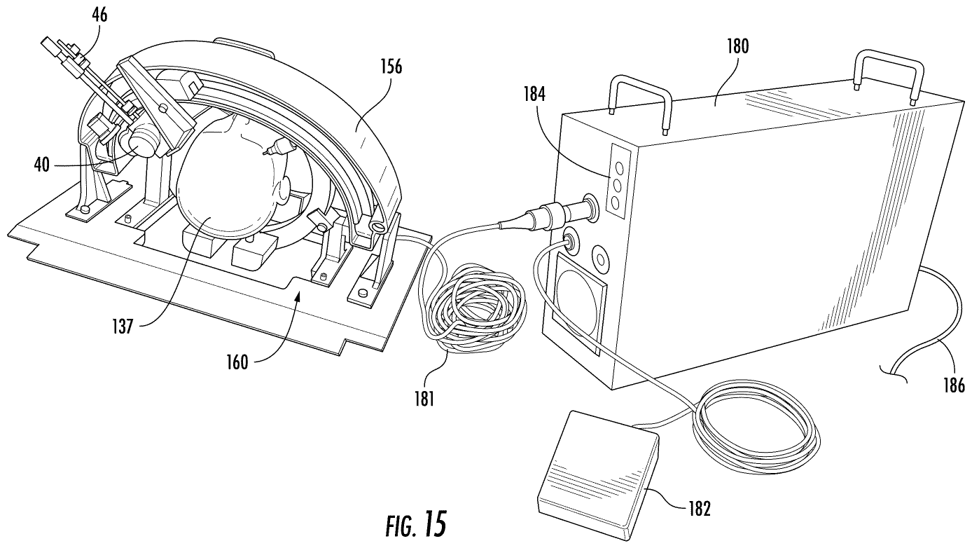

FIG. 15 depicts a representative example configuration of the system and method for localization of a surgical instrument guide wherein a robotic actuated stereotactic alignment guide 156 that is compatible with the MRI environment and the CT environment is coupled to a fiducial-integrated instrument guide 40 as an end effector used for initial registration of the robot to the medical imaging system and for subsequent confirmation of alignment of an instrument 46. The system comprises the robotic stereotactic frame 156 which is coupled to the fiducial-integrated instrument guide as an end effector 40, a base platform 160 with integrated head fixation of the patient 137 that couples with the robotic stereotactic frame 156 and a medical imaging system bed, a control system 180 that is compatible with the MRI environment and can reside inside the MRI scanner room, a footpedal interlock 182 to enable robot motion, a fiberoptic connection 186 to a network interface box that resides outside of the imaging system room (typically beside the imaging system's control console). The network interface box enables coupling the robot controller, surgical navigation, planning and control software, registration software, and the medical imaging system.

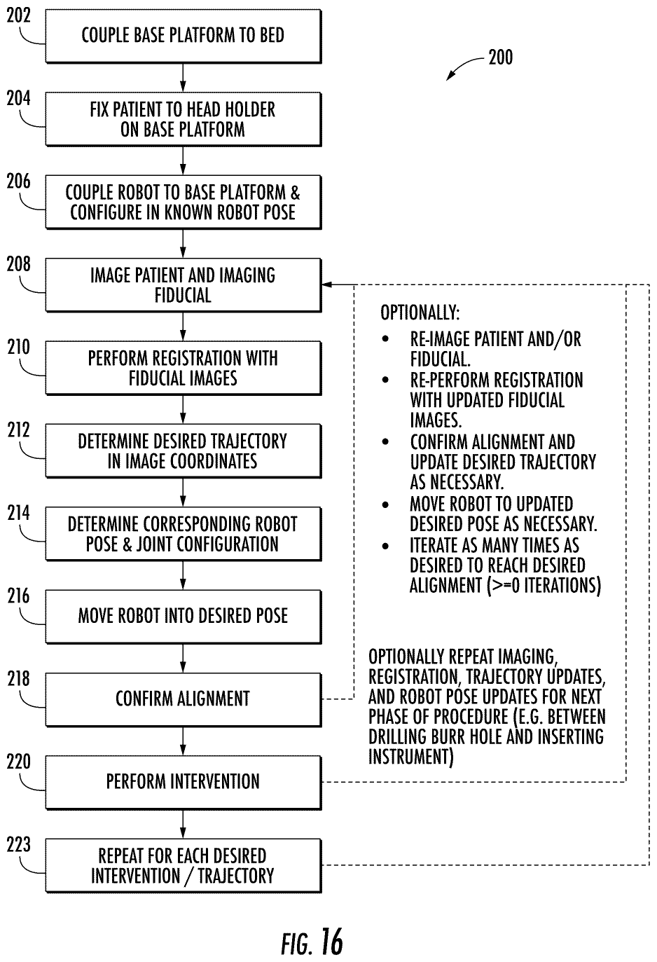

FIG. 16 is a block diagram depicting a workflow for localizing the fiducial-integrated instrument guide according to the present system and method and generally designated at 200. This example is shown for cranial interventions and in particular stereotactic neurosurgery in MRI. It is understood that the system and method for localization of a surgical instrument guide comprises other imaging modalities and other procedures and anatomy that utilize a similar workflow. This system and method also comprises the use of the fiducial-integrated instrument guide used with other instrumentation and alignment devices beyond those detailed herein.

The base platform is fixed to the bed 202 and the patient is fixed to the integrated head holder 204. The sterile field is created and the robotic alignment guide is coupled to the base platform 206 (and by extension the patient and the imaging system bed). Images of the patient are acquired to generate a surgical plan and images of the fiducial are acquired to determine the position and orientation (pose) of the fiducial-integrated instrument guide 208. Coupling that with the known kinematics of the robotic alignment guide in the configuration in which the fiducial was imaged, the pose of the robotic alignment guide with respect to the medical imaging system is determined 210. The surgical plan is then used to determine the required configuration of the robotic alignment guide to align the instrument axis with the target and the necessary insertion depth. The robotic alignment guide is then moved into the desired configuration 212.

Images of the fiducial are again acquired once the robotic alignment guide is in the intended configuration to determine the position and orientation (pose) of the fiducial-integrated instrument guide 214. That is used to virtually project the trajectory of the instrument guide axis as an overlay on the patient images (which may optionally also be reacquired and merged). Optionally, coupling that with the known kinematics of the robotic alignment guide in the configuration in which the fiducial was imaged, the pose of the robotic alignment guide with respect to the medical imaging system is determined again and can be used for re-registration or to confirm there was no unintended or unmodeled motion 216.

This process may be repeated and integrated until satisfactory alignment has been achieved 218. In many cases this will be first performed for aligning a burr hole in the skull, and then the patient re-imaged to assess brain shift and motion or targets and sensitive anatomy, and the planned trajectory for the instrument to be delivered through the burr hole to the target updated accordingly. Then the robotic alignment guide would move to the updated plan and the iterative imaging and adjustment cycle repeated, wherein often no iterations will be required but the workflow enables as many iterations as desired. 220 This process is then repeated for each intended trajectory, which could represent typically two trajectories for bilateral DBS, typically 10-20 trajectories for SEEG mapping electrodes, typically 6-10 trajectories for targeted delivery of biologics, or a number of trajectories as needed for ablation of cancerous or functional targets 223.

One of skill in the art will readily appreciate the teachings herein can be applied to develop multiple devices or use cases embodying the disclosed inventions, and not be restricted to only the specific embodiments, configurations, and use cases explicitly identified.

We claim:

1. A guide for holding an instrument having a longitudinal axis, the instrument guide comprising:
an integrated imaging fiducial for localization, the imaging fiducial including
a plurality of substantially ring-shaped fiducial elements visible in at least one medical imaging modality, each of the plurality of fiducial elements having a central axis that is coaxial with the instrument axis, and
at least one fiducial element visible in at least one medical imaging modality and having a central axis that is not coaxial with the instrument axis.

2. The instrument guide as recited in claim 1, wherein the fiducial elements are visible in magnetic resonance imaging (MRI).

3. The instrument guide as recited in claim 1, wherein the fiducial elements are visible in x-ray based imaging including computed tomography (CT) and related techniques.

4. The instrument guide as recited in claim 1, wherein the fiducial elements are visible in both MRI and CT-based imaging.

5. The instrument guide as recited in claim 1, wherein the fiducial elements are capable of being visualized and used to determine the instrument axis and anticipated trajectory relative to the medical imaging system.

6. The instrument guide as recited in claim 1, further comprising means for determining the position and orientation of the fiducial and associated instrument holder with respect to the medical imaging system.

7. The instrument guide as recited in claim 1, further comprising automatic segmentation of the fiducial elements individually, and fitting each of the fiducial elements to a known configuration relative to a coordinate system defined in the instrument holder to determine the six degree-of-freedom (DOF) position and orientation of the instrument holder respect to the medical imaging system.

8. The instrument guide as recited in claim 1, further comprising automatic segmentation of the fiducial elements, and fitting all of the fiducial elements to a known model of the visible shape to determine the 6-DOF position and orientation of the instrument holder respect to the medical imaging system.

9. The instrument guide as recited in claim 1, further comprising a fiducial-integrated end effector designed to be part of a sterile single patient use kit.

10. The instrument guide as recited in claim 1, wherein the fiducial elements comprise hollow plastic substantially ring-shaped components filled with a fluid visible in MRI, CT, or both MRI and CT.

11. The instrument guide as recited in claim 1, wherein the fiducial elements are capable of being visualized as hyperintense in both MRI and CT.

12. The instrument guide as recited in claim 1, wherein substantially the same image processing localization software algorithm can be used to determine the position and orientation of the instrument holder in both MRI and CT.

13. The instrument guide as recited in claim 1, wherein the fiducial elements are filled with fluid and visible in MRI, and wherein an additional radiopaque material is used to make the fiducial elements visible in CT.

14. The instrument guide as recited in claim 1, further comprising a spacer ring between the MRI visible fiducial elements, wherein the spacer rings are visible in CT.

15. The instrument guide as recited in claim 1, wherein at least part of the housing of the fluid-filled fiducial elements are visible in CT.

16. The instrument guide as recited in claim 1, wherein multi-modality fiducial elements are filled with a fluid visible in MRI and CT.

17. The instrument guide as recited in claim 1, wherein the fiducial elements comprise a material that is safe for use in MRI and does not introduce paramagnetic artifacts.

18. The instrument guide as recited in claim 1, wherein the inner diameter of the fiducial-integrated instrument guide is sufficient to pass a coaxial instrument holder configured for passing an instrument.

19. The instrument guide as recited in claim 1, wherein the inner diameter of the fiducial-integrated instrument guide is sufficient to pass standard stereotactic neurosurgery instrumentation holders and frame adapters.

20. The instrument guide as recited in claim 1, further comprising a linear adjustment parallel to the instrument axis.

21. The instrument guide as recited in claim 20, wherein the linear adjustment has discrete intervals.

22. The instrument guide as recited in claim 20, wherein the linear adjustment is continuously variable along a linear guide.

23. The instrument guide as recited in claim 20, wherein the linear adjustment is at least one of actuated and encoded.

24. The instrument guide as recited in claim 1, wherein the fiducial-integrated instrument holder comprises an end effector of an actuated alignment device.

25. The instrument guide as recited in claim 24, wherein the fiducial is used to determine position and orientation of the end effector relative to the medical imaging system.

26. The instrument guide as recited in claim 24, wherein the position and orientation of the end effector relative to the medical imaging system in combination with kinematics information of the actuated alignment device are used to localize the coordinate system position and orientation of the actuated alignment device relative to that of the imaging system and the patient.

27. The instrument guide as recited in claim 24, wherein the position and orientation of the fiducial-integrated instrument guide end effector is used for assessing the trajectory of the instrument after alignment.

28. The instrument guide as recited in claim 24, wherein the position and orientation of the fiducial-integrated instrument guide end effector is used for repeated intraoperative registration of the alignment device to the imaging system.

29. The instrument guide as recited in claim 24, wherein the position and orientation of the fiducial-integrated instrument guide end effector is used for identifying errors in alignment including alignment device positioning, registration errors, imaging distortion, target motion, or patient motion.
